(19) United States
(12) Patent Application Publication
(10) Pub. No.: US 2013/0266580 A1
(43) Pub. Date: Oct. 10, 2013

(54) Methods and Compositions for Using Insulin-Like Growth Factor Binding Protein 6 in Treating and Diagnosing Diabetes

(71) Applicant: GEORGIA REGENTS UNIVERSITY, Augusta, GA (US)

(72) Inventor: Jin-Xiong She, Martinez, GA (US)

(73) Assignee: Georgia Regents University, Augusta, GA (US)

(21) Appl. No.: 13/851,735

(22) Filed: Mar. 27, 2013

Related U.S. Application Data
(63) Continuation of application No. PCT/US2013/030577, filed on Mar. 12, 2013.
(60) Provisional application No. 61/621,289, filed on Apr. 6, 2012.
(60) Provisional application No. 61/662,098, filed on Jun. 20, 2012.

(51) Int. Cl. G01N 33/68

(52) U.S. Cl. CPC G01N 33/6893
USPC 424/158.1; 435/7.92; 436/501; 435/6.12

(57) ABSTRACT

Methods and compositions for treating, detecting, diagnosing or assisting in the diagnosis of diabetes or diabetes-related complications are provided. Insulin-growth factor binding protein 6 (IGFBP6) has been determined to be a biomarker for type 1 diabetes (T1D) as well as for certain T1D complications such as nephropathy, retinopathy, and blindness. Compositions that prevent expression of IGFBP6 or block binding of IGFBP6 with its target, insulin-growth factor II (IGF-II), can be used to treat one or more symptoms or pathological processes of diabetes. Assaying for levels of IGFBP6 can be used to monitor disease progression, determine disease severity, and determine risk of developing diabetes or diabetes-related complications.

### CROSS REFERENCE TO RELATED APPLICATIONS

This application is a continuation of International Application No. PCT/US2013/30577 filed in United States Receiving Office for the Patent Cooperation Treaty on Mar. 12, 2013, by Jin-Xiong She, which claims benefit of and priority to U.S. Provisional Application Nos. 61/621,289, filed Apr. 6, 2012 and 61/662,098, filed Jun. 20, 2012, which are incorporated by reference in their entirety.

### STATEMENT REGARDING FEDERALLY SPONSORED RESEARCH OR DEVELOPMENT

This invention was made with Government Support under Agreements 4R33HD050196, 4R33-DK069878 and 2RO1HD37800 awarded by the National Institutes of Health. The Government has certain rights in the invention.

### FIELD OF THE INVENTION

The invention is generally directed to compositions and methods related to the treatment of diabetes.

### BACKGROUND OF THE INVENTION

About 3 million people in the world have Type 1 Diabetes (T1D). Every year, approximately 15,000 children and 15,000 adults are diagnosed with T1D. T1D is an autoimmune disease that destroys beta cells in the pancreas. The destruction of beta cells results in little to no insulin production. Because insulin is involved in the pathway of converting glucose to energy, a reduction in insulin production leads to a build up of glucose which is harmful to the body. At this time, there are no known therapies to prevent T1D and no known treatments to cure T1D.

Many complications are associated with T1D. Preventing or identifying these complications at an early stage can be critical in the treatment of T1D. In general, subjects with T1D try to control the disease and treat the complications associated with T1D. However, not identifying many of the associated complications at an early stage can lead to long term, irreparable damage. Accordingly, the need exists for improved methods for detecting, predicting and treating T1D and its associated complications.

It is an object of the invention to provide compositions and methods that can be used to detect or predict the severity of or the risk of developing T1D.

It is another object of the invention to provide methods and compositions for identifying therapeutics for treating or preventing T1D.

It is still another object of the invention to provide methods and compositions for treating diabetes, preferably diabetes mellitus type 1.

### SUMMARY OF THE INVENTION

Methods and compositions for the diagnosis, prognosis or treatment of diabetes are provided. The methods include assaying levels of insulin-like growth factor binding protein 6 (IGFBP6) in the treatment of diabetes, preferably diabetes mellitus type 1 (T1D). The levels of IGFBP6 can be used to determine the risk of developing diabetes, determine the severity of diabetes, determine the effectiveness of a treatment for diabetes, and to identify lead compounds for treating diabetes. Exemplary methods include assaying for serum levels of IGFBP6 of a subject, wherein an increase in IGFBP6 levels in the sample compared to a control level is an indication that the subject has diabetes.

Methods of determining the risk of a subject for developing diabetes include assaying for levels of IGFBP6 in a sample from a subject, wherein an increase in IGFBP6 levels in the sample compared to a control level is an indication that the subject is at risk of developing T1D.

Further provided are methods of selecting a therapy for a subject. Different therapeutics or treatments can be selected based on the levels of IGFBP6 present in a sample from a subject. As an example, an increase in IGFBP6 levels in the sample compared to a control level can indicate that a therapy is needed. The therapy can be a preventative treatment or a treatment to reduce the symptoms or complications from the diabetes. Thus, a subject with diabetes having an increase in IGFBP6 can be identified as a subject that needs a specific therapy to treat the diabetes or a diabetes-related complication such as nephropathy.

### BRIEF DESCRIPTION OF THE DRAWINGS

FIGS. 1A-1H shows regression graphs (dot plots) of ng/ml of IGFBP6 versus age. The graphs show the correlation between serum IGFBP6 levels and age of subjects. Relative levels of IGFBP6 as a function of age in the total control group (FIG. 1a, β=0.013, p=3×10⁻⁹), female controls (FIG. 1b, β=0.009, p=0.006), male controls (FIG. 1 c, β=0.02, p=6.6×10⁻¹¹), and regression lines for control males and females on same plot (FIG. 1d). Relative levels of IGFBP6 as a function of age in the total T1D group (FIG. 1 e, β=0.018, p=1×10⁻¹⁶), female T1D (FIG. 1f, β=0.016, p=1×10⁻¹³), male T1D (FIG. 1g, β=0.021, p=8.3×10⁻¹⁴), and regression lines for T1D males and females on same plot (FIG. 1h).

FIGS. 2A-2D show regression plots (dot plots) of IGFBP6 levels versus the duration of disease for all T1D patients (FIG. 2a; β=0.023, p=1×10⁻¹⁶), female T1D (FIG. 2b, β=0.024, p=3.1×10⁻¹²), male T1D (FIG. 2c, β=0.023, p=1×10⁻¹¹), and regression lines for both genders on same plot (FIG. 2d).

FIGS. 3A and 3B show dot plots of serum IGFBP6 (ng/ml) versus control and T1D subjects. The graphs show the distribution of serum IGFBP6 levels in T1D and control groups after matching for sex and age. Dash lines mark the 95^{th} percentile value in T1D patients. Solid lines and values on the right side represent the mean values of serum IGFBP6 in the respective groups. IGFBP6 serum levels in total T1D and control subjects are shown in FIG. 3a. Separation of both T1D and control groups into female and male subjects is shown in FIG. 3b.

FIG. 4 shows boxplots of serum IGFBP6 (ng/ml) versus T1D without any complications (NoC) and T1D with complications (Comp).

### DETAILED DESCRIPTION OF THE INVENTION

### I. Definitions

As used herein, the term “subject” means any individual who is the target of administration. The subject can be a vertebrate, for example, a mammal. Thus, the subject can be a human. The term does not denote a particular age or sex. Thus, adult and newborn subjects, whether male or female, are intended to be covered. A patient refers to a subject afflicted with a disease or disorder. The term “patient” includes human and veterinary subjects.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “prognosis” encompasses predictions about the likely course of disease or disease progression, particularly with respect to likelihood of disease remission, disease relapse, and death. “Good prognosis” refers to the likelihood that a patient afflicted with diabetes, particularly T1D, will remain without diabetes-related complications. “Poor prognosis” is intended to mean the likelihood of diabetes-related complications or death.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### II. Insulin-Like Growth Factor Binding Proteins

Insulin and insulin-like growth factors (IGFs) are central hormones in regulating metabolism, glucose uptake, glycogen synthesis, lipogenesis and prevention of proteolysis [Firth S M and Baxter R C. Endocr Rev 2002; 23(6): 824-854]. The IGF axis is a complex system composed of two mitogenic ligands, IGF-I and IGF-II, two receptors, IGF-IR and IGF-IIR and binding proteins [Le Roith D. Exp Diabesity Res 2003; 4(4): 205-212]. Insulin-like growth factors, IGF-I and IGF-II are single chain peptides with significant homology to insulin. IGF-II has been implicated in mitosis, growth, and organ development by paracrine and endocrine pathways in cell culture studies and animal models [Smith T J. Pharmacol Rev 2010; 62(2): 199-236; Hwa V, et al. Endocr Rev 1999; 20(6): 761-787]. IGF-II also has insulin-like metabolic effects and overproduction of IGF-II in certain pancreatic and non-pancreatic cancers has been linked to severe refractory hypoglycemia [Korevaar T I M, et al. QJM: Int J Med 2012; Barra et al. Case Report Med 2010; 2010: 684045]. The actions of IGFs may be mediated through binding to the IGF-I/II receptor (a homologue of insulin receptor) and/or to the insulin receptor itself [Firth S M and Baxter R C. Endocr Rev 2002; 23(6): 824-854, Sima A A, et al. Exp Diabesity Res 2003; 4(4): 235-256].

The bioavailability of the IGFs to its receptors is controlled by IGF binding proteins (IGFBPs), which represent an important link between the insulin and IGF system. IGFBPs belong to a superfamily of 15 homologous proteins, present in serum, other biological fluids and tissue extracts [Hwa V, et al. Endocr Rev 1999; 20(6): 761-787]. Although IGFBPs are globally distributed in all tissues and cells, the majority of circulating levels of IGFBPs are produced by the liver under regulation of IGFs and insulin [Rajaram S, et al. Endocr Rev 1997; 18(6): 801-831]. IGFBP3 is the most abundant protein of the family followed by IGFBP2 and IGFBP1. IGFBP4 and IGFBP5 represent a smaller portion of the IGFBP family in the circulation [Rajaram S, et al. Endocr Rev 1997; 18(6): 801-831]. IGFBP6 is the sixth member of the IGFBP family and has a preference towards binding to IGF-II in a specific manner than IGFBP1 [Bach L A, et al. J Biol Chem 1993; 268(13): 9246-9254]. Four of the IGFBPs, (IGFBP-1, IGFBP2, IGFBP3 and IGFBP5) have been implicated in T1D, prevention of obesity and induction of insulin resistance in both animal models and human subjects [Mohamed-Ali V, et al. Clin Endocrinol (Oxf) 1999; 50(2): 221-228; Feldmann B, et al. Growth Horm IGF Res 2000; 10(1): 53-59; Ruan W and Lai M. Acta Diabetol 2010; 47(1): 5-14; Wheatcroft S B and Kearney M T. Trends Endocrinol Metab 2009; 20(4): 153-162]. These IGFBPs are identified as surrogate markers for metabolic syndrome, cardiovascular disease and cancers in several studies. IGFBPs are also known to be involved in augmentation of micro- and macro-vascular complication of diabetes [Mohamed-Ali V, et al. Clin Endocrinol (Oxf) 1999; 50(2): 221-228; Feldmann B, et al. Growth Horm IGF Res 2000; 10(1): 53-59; Bayes-Genis A, et al. Circ Res 2000; 86(2): 125-130; Sima A A, et al. Exp Diabesity Res 2003; 4(4): 235-256; Ruan W and Lai M. Acta Diabetol 2010; 47(1): 5-14; Livingstone C and Ferns G. A. Br J Diabetes Vasc Dis 2003; 3: 326-331; Smith T J. Pharmacol Rev 2010; 62(2): 199-236; Wheatcroft S B and Kearney M T. Trends Endocrinol Metab 2009; 20(4): 153-162].

### III. Type 1 Diabetes

Type 1 diabetes (T1D) is an autoimmune disease that attacks and destroys beta cells in the pancreas. The beta cells are located within small islands of endocrine cells called the pancreatic islets. Beta cells normally produce insulin which is a hormone that helps move the glucose (i.e., sugar) contained in food into cells throughout the body. The body uses glucose for energy. However, when the beta cells are destroyed, no insulin can be produced. The depletion of insulin results in glucose accumulating in the blood which can lead to serious damage to all the organ systems of the body.

Therefore, being able to determine the severity or risk of diabetes, particularly T1D, is essential. The increased levels of IGFBP6 in individuals with diabetes, allows for a biomarker that can quickly and easily be determined.

T1D Complications

T1D can affect many major organ systems in the body. In particular, the heart, blood vessels, nerves, eyes and kidneys can all be affected either individually or in combination with each other. Complications from T1D include, but are not limited to, cardiovascular problems (i.e. coronary artery disease with angina, heart attack, stroke, atherosclerosis and high blood pressure), neuropathy, nephropathy, eye problems (i.e. diabetic retinopathy, blindness, cataracts, glaucoma), foot damage, skin problems (i.e. bacterial and fungal infections), osteoporosis, pregnancy complications (i.e. miscarriage, stillbirth, birth defects, preeclampsia), and hearing problems.

### IV. Diagnostic/Prognostic Methods

Methods of predicting, prognosticating and monitoring are provided. The presence, absence, increase, or decrease of IGFBP6 in a sample from a subject can be used to predict the risk of diabetes, determine the severity of the diabetes, or monitor the disease complications. In a preferred embodiment, there is an increase in IGFBP6 compared to a reference level. In some embodiments the subject has never been diagnosed with diabetes. In some embodiments the subject has been diagnosed with T1D.

The methods can include administering a therapeutic in an effective amount to treat the diabetes or a diabetic complication based on the increase in IGFBP6 compared to a reference level.

In some embodiments, the risk of diabetes and the severity of diabetes can be determined using a combination of IGFBP6 and other known diabetes biomarkers. Other diabetes biomarkers include, but are not limited to, 1,5 Anhydroglucitol, islet cell antibodies, insulin autoantibodies, glutamic acid decarboxylase autoantibodies, tyrosine phosphatases IA-2 and IA-2β autoantibodies, adiponectin, CRP, IL-1 receptor antagonist and ferritin.

In some embodiments, IGFBP6 can be combined with classic diabetes risk factors, such as BMI and blood glucose levels, to predict risk and severity of disease.

Provided herein is a method for evaluating the prognosis of a subject with T1D which involves detecting at least the IGFBP6 biomarker in the subject, wherein the presence, level, amount, or a combination, of IGFBP6 is indicative of the prognosis of the subject.

An increase in IGFBP6 levels can mean a significant increase using standard statistical formulas. An increase can also mean levels being 5% or greater increase compared to the control levels.

A. Methods of Determining the Severity of Diabetes

The severity of diabetes in individual subjects can vary significantly. The severity can depend on how early the diabetes was diagnosed or treated. The severity of diabetes can depend on the severity of the complications associated with diabetes. For example, a diabetic exhibiting no significant complications is not as severe as a diabetic with one or more diabetes-related complications or a diabetic with hearing loss may not be considered as severe as a diabetic with nephropathy.

Determining the severity of the diabetes allows for the subject's treatment plan to be adjusted. If IGFBP6 is increased in a sample from a subject compared to a reference level, the disease can be considered more severe and a specific treatment plan can be implemented immediately. The medical professional caring for the subject can determine the best approach for treating the diabetes.

1. Assaying for Levels of IGFBP6

Methods of determining, examining or measuring expression levels of IGFBP6 can be performed by detecting nucleic acids or protein.

a. Assaying Protein Levels

Determining protein levels of IGFBP6 in a sample typically, but not exclusively, involve the use of agents that bind to the relevant proteins. Common protein binding agents are antibodies and, most ideally, monoclonal antibodies which, advantageously, have been labeled with a suitable tag whereby the existence of the bound antibody can be determined.

Assay techniques for identifying or detecting proteins are well known to those skilled in the art and are used every day by workers in the field of clinical diagnostics. Such assay techniques can be applied by the skilled worker to utilize the invention. Examples of protein detection assays include, but are not limited to, immunoassays such as enzyme-linked immunosorbant assays (ELISA), western blots, dot blots, radioimmunoassay (RIA), fluoroimmunoassay (FIA), immunoprecipitation and the like.

b. Assaying RNA Levels

Techniques available for measuring RNA content are well known in the art and routinely practiced by those in the clinical diagnostics field. Such techniques can include reverse transcription of RNA to produce cDNA and an optional amplification step followed by the detection of the cDNA or a product thereof. Examples of detecting nucleic acids include but are not limited to PCR, reverse-transcription PCR, real-time quantitative PCR (Jiang et al 2003a and 2004. Jiang W G, Watkins G, Lane J, Douglas-Jones A, Cunnick G H, Mokbel M, Mansel R E. Prognostic value of Rho familty and rho-GDIs in breast cancer. Clinical Cancer Research, 2003a, 9, 6432-6440; Jiang W G, Watkins G, Fodstad O, Douglas-Jones A, Mokbel K, Mansel R E. Differential expression of the CCN family members Cyr61 from CTGF and Nov in human breast cancer. Endocrine Related Cancers, 2004, 11: 781-791, northern blot, southern blot, and dot blots.

The RNA can be isolated from the sample before the levels are measured. Methods of isolating RNA are well known in the art.

c. Reference Levels and Control

The level of expression of a given molecular marker, such as IGFBP6, can be determined having regard to a reference gene or protein (such as, but not limited to, GAPDH or actin) within a sample. The reference gene or protein can either be a measured in a sample from the same subject being measured for IGFBP6 or from an age and sex-matched control sample. In some embodiments, the control sample is a subject with diabetes but without any diabetes-related complications.

In some embodiments, the control sample is a sample from a healthy individual. Thus increased expression of IGFBP6 in a sample from a subject refers to an increase in expression with regard to the expression of a control gene or protein either in the same sample or in a different sample.

d. Samples

IGFBP6 levels are determined or measured in a sample from a subject. The sample can be a biological fluid or cell sample. The biological fluid includes but is not limited to serum, blood, urine, cerebrospinal fluid, or saliva. The cell sample can include any cell type, such as lymphocytes or endothelial cells, or cell lysates.

B. Methods of Determining the Risk of Diabetes

Determining the risk of diabetes can be accomplished based on the levels of IGFBP6 in a sample from a subject. If IGFBP6 levels in a sample from a subject are the same or in a similar range as IGFBP6 levels from known healthy individuals then the subject can be determined to have little or no risk of developing diabetes. If IGFBP6 levels in a sample from a subject are higher than IGFBP6 levels from known healthy individuals then the subject can be determined to have an increased risk of developing diabetes.

A determination of little to no risk of developing diabetes can mean that the subject has less than a 20%, 15%, 10%, 5%, 4%, 3%, 2%, or 1% chance of developing diabetes. The time between the determination of the risk or severity of diabetes or a diabetes-related complication and the time when the diabetes or the complication occurs can vary. In some embodiments the time from determination to the disease or complication can be days, weeks or months. In some embodiments, a determination of having little to no risk of developing diabetes is valid only for a 5 year, 4 year, 3 year, 2 year, 1 year or 6 month time frame from the date of determination. Therefore, an individual may need to have IGFBP6 levels examined on a regular basis.

A determination of an increased risk of developing diabetes can mean that the subject has greater than a 30%, 40%, 50%, 60%, 70%, 80%, 90% or 100% chance of developing diabetes. In some embodiments, the subject may never develop diabetes because the subject can alter their lifestyle after the determination which thereby will decrease their risk of developing diabetes.

In some embodiments, the determination of the risk of developing diabetes is performed by measuring levels of IGFBP6 along with considering other known risk factors, such as diet and family history.

The levels of IGFBP6 can also be used to determine the risk of developing one or more diabetes-related complications in those subjects already diagnosed with diabetes.

C. Methods of Selecting a Therapy

The levels of IGFBP6 can be used to select a specific therapy. IGFBP6 levels can be associated with different diabetes-related complication or disease severities.

If it is determined that there is an increase in IGFBP6 in a sample from a subject, then a specific therapy can be initiated. For instance, the increased levels can indicate that the subject is suffering from nephropathy, neuropathy or a diabetic ulcer. Known medications for those complications can be prescribed to the subject. The increase in IGFBP6 can occur just prior to physical symptoms of disease severity and therefore determining the levels of IGFBP6 can provide a mechanism for prophylactic treatment.

In some embodiments, a decrease in IGFBP6 in a sample from a subject can indicate that a lower dose or no medication is necessary at that time. A decrease in IGFBP6 can be determined based on comparison to the levels of IGFBP6 from a sample taken during disease flare up.

### IV. Screening Methods

Also provided are methods of identifying agents for use in treating diabetes. The screening methods involve contacting a sample containing IGFBP6 with a candidate agent and assaying for the presence of IGFBP6 or the ability of IGFBP6 to bind to IGF-II. A decrease in the presence of IGFBP6 indicates that the candidate agent inhibited the expression of IGFBP6 and can be an effective agent for use in treating T1D. A decrease in the ability of IGFBP6 to bind to IGF-II indicates that the candidate agent blocked or prevented binding of IGFBP6 with IGF-II and can be an effective agent for use in treating T1D. As used herein, the ability of an agent to “treat” T1D includes the ability of the agent to prevent or reduce the complications related to T1D.

In some embodiments, the IGFBP6 present in the sample used for the screening method is the IGFBP6 protein. In some embodiments, the IGFBP6 is the gene encoding IGFBP6 or the IGFBP6 transcript.

A decrease in IGFBP6 can be determined by comparing to IGFBP6 levels in a control. Controls for the screening methods can include a sample containing IGFBP6 that does not receive the candidate agent or is only contacted with the solution the candidate agent is present in but without the candidate agent.

The presence, absence or decrease in IGFBP6 can be assayed with common immunological assays known in the art. The ability of IGFBP6 to bind to IGF-II after encountering a therapeutic candidate agent can be assayed by techniques known to those of skill in the art. For example, a sample containing IGFBP6 that is contacted with a candidate agent can later be contacted with IGF-II. The IGF-II can be bound to a surface such as beads. The IGFBP6/candidate agent solution can be added to the beads containing IGF-II. If the IGFBP6 is bound to the candidate agent, then IGFBP6 will not bind to IGF-II. If the candidate agent does not block IGFBP6 from binding to IGF-II then IGFBP6 will bind to the IGF-II and the IGFBP6-IGF-II complex can be separated from everything else using the beads that are bound to the IGF-II. The IGFBP6-IGF-II can then be assayed to confirm that both IGFBP6 and IGF-II are present which indicates that the candidate agent is not a good therapeutic. One of skill in the art would understand all of the possible ways to test for binding of IGFBP6 to IGF-II.

A. Candidate Agents

The candidate agents can be proteins, nucleic acids or compounds. Candidate agents can be identified from large libraries of natural products or synthetic (or semi-synthetic) extracts or chemical libraries according to methods known in the art. Those skilled in the field of drug discovery and development will understand that the precise source of extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that blocks binding of IGFBP6 to IGF-II. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents can have cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of prokaryotic and eukaryotic proteins can be made for screening using the methods herein. The libraries can be bacterial, fungal, viral, and vertebrate proteins, and human proteins.

### V. Methods of Treating

Methods of treating diabetes type 1 in a subject by administering a composition to the subject in an amount effective to reduce serum levels of IGFBP6 relative to serum levels of IGFBP6 of the subject prior to treatment are disclosed. The reduction in serum levels of IGFBP6 can treat one or more symptoms of diabetes or diabetic complications.

Methods of treating diabetes in a subject can also include administering a composition to the subject in an amount effective to reduce serum levels of IGFBP6 wherein after administration of the composition the serum levels of IGFBP6 can be assayed and treatment can be discontinued if IGFBP6 serum levels are normal. Normal IGFBP6 levels can be those levels that are the same as serum IGFBP6 levels in the subject before the onset of diabetes or before the onset of the diabetic complication. In some embodiments, normal levels of serum IGFBP6 can be those levels determined to be normal or standard in healthy individuals.

The methods of treating diabetes or diabetic complications can be performed on subjects that were selected for based on their risk of developing diabetes or diabetic complications.

A. Treating with IGF-II

Disclosed are methods of treating diabetes, particularly type 1 diabetes. The methods can include administering an effective amount of IGF-II to a subject with type 1 diabetes. In some embodiments the subject has elevated IGFBP6. In some embodiments the subject can have diabetes-related complications.

IGF-II is known to have anti-apoptotic effects. Because IGFBP6 can bind to IGF-II, the elevated levels of IGFBP6 can lead to binding of IGFBP6 and IGF-II. IGF-II bound to IGFBP6 can have reduced anti-apoptotic effects leading to increased apoptosis in selected tissues or organs when IGFBP6 is bound to IGF-II. Therefore, treating a subject having elevated levels of IGFBP6 with IGF-II can result in the presence of active IGF-II and thus anti-apoptotic effects. In other words, the administered IGF-II can replace the native IGF-II and provide the same function. Or the administered IGF-II can bind to IGFBP6 leaving the native IGF-II available to provide its normal biological functions. Treating with IGF-II can lead to reduced IGFBP6 damage.

In some embodiments, the IGF-II is recombinant. The IGF-II can be wild type or can be a mutated form of IGF-II. In some embodiments, only a fragment of IGF-II is administered. The fragments of IGF-II must retain their ability to provide therapeutic effects, such as anti-apoptotic effects. In some embodiments, a nucleic acid encoding any of the disclosed IGF-II can be administered to a subject.

B. Treating with an Inhibitor of IGFBP6

Disclosed are methods of treating diabetes, particularly type 1 diabetes, by administering an effective amount of an inhibitor of IGFBP6 to a subject having type 1 diabetes. The subject can have elevated levels of IGFBP6. In some embodiments the subject can be experiencing diabetes-related complications.

Because the binding of IGFBP6 to IGF-II can result in the inhibition of the normal biological activity of unbound IGF-II, blocking or preventing the binding of IGFBP6 with IGF-II can be therapeutic. Therefore, IGF-II peptide mimics that contain the IGFBP6 binding site can be used. As long as the IGF-II peptide mimic binds to or blocks IGFBP6 from binding to native IGF-II, the peptide mimic can have therapeutic effects.

Other inhibitors that can be used to prevent the binding of IGFBP6 with IGF-II include antibodies or antibody fragments. For example, an antibody or antibody fragment that binds to IGFBP6 and prevents IGFBP6 from binding to IGF-II can be used as an inhibitor. Antisense RNA or siRNA specific for IGFBP6 can also be used. These RNAs can bind to IGFBP6 RNA and prevent IGFBP6 protein from being made. In some instances, expression of the antisense or siRNA can be turned and off in order to allow IGFBP6 protein expression at certain times.

Compounds, proteins or nucleic acids that prevent IGFBP6 from binding to IGF-II can be used as an inhibitor.

C. Combination Therapy

In certain embodiments, the methods for treating diabetes include administering additional therapeutic agents that may or may not inhibit or reduce IGFBP6. For example, the disclosed therapeutic agents can be administered in combination with known diabetes therapeutics. The combination therapeutic can be used for treating one or more diabetes-related complications.

Representative diabetes therapeutics include, but are not limited to insulin and insulin derivatives, somatostatin and somatostatin analogs, ACE inhibitors, angiotensin II receptor blockers, aspirin, metoclopramide or erythromycin, pain relievers, and statins.

D. Pharmaceutical Compositions

The IGFBP6 inhibitors disclosed herein can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution or suspension (for example, incorporated into microparticles, liposomes, or cells). These may be targeted to a particular cell type via antibodies, receptors, or receptor ligands. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

E. Therapeutic Administration The disclosed IGFBP6 inhibitors, including pharmaceutical compositions, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions can be administered prophylactically to patients or subjects who are at risk for T1D. Thus, the method can further comprise identifying a subject at risk of T1D prior to administration of the disclosed IGFBP6 inhibitors.

The exact amount of the compositions required will vary from subject to subject, depending on the species, age, weight and general condition of the subject, the severity of the diabetes or diabetes-related complications being treated, the particular inhibitor used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Therapeutic Antibodies, Chernajovsky et al., Springer-Verlag, Berlin Heidelberg (2008); Handbook of Monoclonal Antibodies, Ferrone et al., eds., Noges Publications, Park Ridge, N.J., (1985) ch. 22 and pp. 303-357. A typical daily dosage of the antibody used alone might range from about 1 μg/kg to up to 100 mg/kg of body weight or more per day, depending on the factors mentioned above.

### EXAMPLES

### Example 1

### Serum Insulin-Like Growth Factor Binding Protein 6 (IGFBP6) is Increased in Patients with Type 1 Diabetes and its Complications

### Methods

Serum Samples

Blood samples were collected from participants of Prospective Assessment in Newborns of Diabetes Autoimmunity (PANDA) [Carmichael S K, et al. Genet Med 2003; 5(2): 77-83] study, after informed consent. The blood was allowed to clot at room temperature for 30 minutes and then centrifuged at 2000×g for 10 minutes in a serum separator tubes. Serum was harvested, aliquoted and stored at −80° C. for future use. All subjects were recruited in Georgia, USA, mainly in the Atlanta and Augusta areas.

Serum samples from 697 T1D patients and 681 healthy controls were aliquoted randomly into wells of 96 well v-bottom plates and each plate contained similar numbers of samples from T1D patients and controls. From blood collection to the assay, none of the samples had more than three freeze/thaw cycles and each sample usually had only one freeze/thaw cycle.

Luminex Assay for IGFBP6

IGFBP6 in serum was measured using a Luminex bead array kit from Millipore (Millipore Inc, Billerica Mass., USA) according to manufacturer's protocol. The kit is based on sandwich immuno-assay, which consists of dyed microspheres conjugated with a specific monoclonal capture antibody. Briefly serum samples were incubated with the antibody-coupled microspheres, and later on with biotinylated detection antibody before the addition of streptavidin-phycoerythrin. The captured bead-complexes were then read by a FLEXMAP3D (Luminex, TX, USA) with the following instrument settings: events/bead: 50, minimum events: 0, Flow rate: 60ul/min, Sample size: 50ul, discriminator gate: 8000-13500.

Statistical Analysis

Median fluorescence intensities were converted to concentrations using a 4-parameter logistic regression fit to the standard curve with known concentration included on each plate using a serial dilution series, as previously described [Zhi W, et al. Mol Cell Proteomics 2011]. All further statistical analyses were performed using log 2 transformed concentrations to achieve more normal distributions. Linear regression analysis was used to determine the effect of age, sex and duration of disease on IGFBP6 levels. Since there was a significant effect of age and sex on the IGFBP6 levels, case-control matching was performed with respect to age, and sex using the “matching” R package [Sekhon J S and Grieve R D. Health Econ 2011]. Potential differences between T1D patients, healthy controls and subsets based on gender and specific complications were initially examined using a paired t-test. To estimate the relative risk of diabetes at different protein concentrations, conditional logistic regression was performed on matched paired data. The odds ratios (OR) and 95% confidence intervals (CI) were computed. To investigate the association between serum IGFBP6 levels and the diabetic complications, the samples from the T1D patients with a particular complication were compared with the samples from the T1D patients without complications. Matching was performed with respect to age, sex, and duration of diabetes. Conditional logistic regression was performed to estimate the relative risk of a particular complication at different protein concentrations. IGFBP6 protein levels were used as continuous as well as discrete variable (after dividing into 4 quartiles). All statistical analyses were performed using the R language and environment for statistical computing (R version 2.12.1; R Foundation for Statistical Computing; www.rproject.org) [R Development Core Team. R: A language and environment for statistical computing. 2010].

### Results

Serum IGFBP6 levels were measured using a Luminex assay for 697 T1D patients and 681 control subjects. The sex and age distributions of the samples before and after matching are presented in Table 1.

TABLE 1
Mean values of age, sex, and T1D duration in cases and controls
before and after multivariate matching.

| | | | | After Matching | |
|---|---|---|---|---|---|
| | | Before Matching | | Con- | |
| Group | | Controls | Cases | trols | Cases |
| T1D | Age (years) | 22.98 | 29.15 | 28.06 | 29.15 |
| | Sex (Female %) | 53.3% | 53.2% | 53.2% | 53.2% |
| Nephropathy | Age (years) | 22.23 | 49.84 | 49.38 | 49.84 |
| | Sex (Female %) | 53.4% | 52.8% | 52.8% | 52.8% |
| | T1D Duration | 9.26 | 31.11 | 29.22 | 31.11 |
| Photo- | Age (years) | 22.23 | 50.95 | 50.54 | 50.95 |
| coagulation | Sex (Female %) | 53.4% | 49.1% | 49.1% | 49.1% |
| | T1D Duration | 9.26 | 34.63 | 33.94 | 34.63 |
| Retinopathy | Age (years) | 22.23 | 51.46 | 50.84 | 51.46 |
| | Sex (Female %) | 53.4% | 57.5% | 57.5% | 57.5% |
| | T1D Duration | 9.26 | 32.47 | 31.50 | 32.47 |
| HTN | Age (years) | 22.23 | 51.41 | 51.15 | 51.41 |
| | Sex (Female %) | 53.4% | 52.1% | 52.1% | 52.1% |
| | T1D Duration | 9.26 | 26.94 | 26.18 | 26.94 |
| Blindness | Age (years) | 22.23 | 48.61 | 50.20 | 48.61 |
| | Sex (Female %) | 53.4% | 53.3% | 53.3% | 53.3% |
| | T1D Duration | 9.26 | 33.46 | 32.05 | 33.46 |
| Foot_Ulcer | Age (years) | 22.23 | 52.61 | 51.63 | 52.61 |
| | Sex (Female %) | 53.4% | 60.0% | 60.0% | 60.0% |
| | T1D Duration | 9.26 | 37.86 | 34.28 | 37.86 |
| CAD | Age (years) | 22.23 | 58.22 | 56.87 | 58.22 |
| | Sex (Female %) | 53.4% | 48.6% | 48.6% | 48.6% |
| | T1D Duration | 9.26 | 30.68 | 28.95 | 30.68 |
| Amputation | Age (years) | 22.23 | 51.97 | 52.52 | 51.97 |
| | Sex (Female %) | 53.4% | 50.0% | 50.0% | 50.0% |
| | T1D Duration | 9.26 | 30.85 | 31.34 | 30.85 |
| Neuropathy | Age (years) | 22.23 | 54.96 | 53.82 | 54.96 |
| | | 0.53 | 0.57 | 0.57 | 0.57 |
| | T1D Duration | 9.26 | 28.24 | 26.87 | 28.24 |
| Dyslipidemia | Age (years) | 22.23 | 47.56 | 47.66 | 47.56 |
| | Sex (Female %) | 53.4% | 55.4% | 55.4% | 55.4% |
| | T1D Duration | 9.26 | 22.81 | 22.31 | 22.81 |

For T1D group, cases are T1D patients, controls are AbN controls, matching was performed with respect to age, and sex.
For Complications, cases are the T1D patients with a particular complication, controls are T1D patients without any complications. Matching was performed with respect to age, sex and T1D duration.

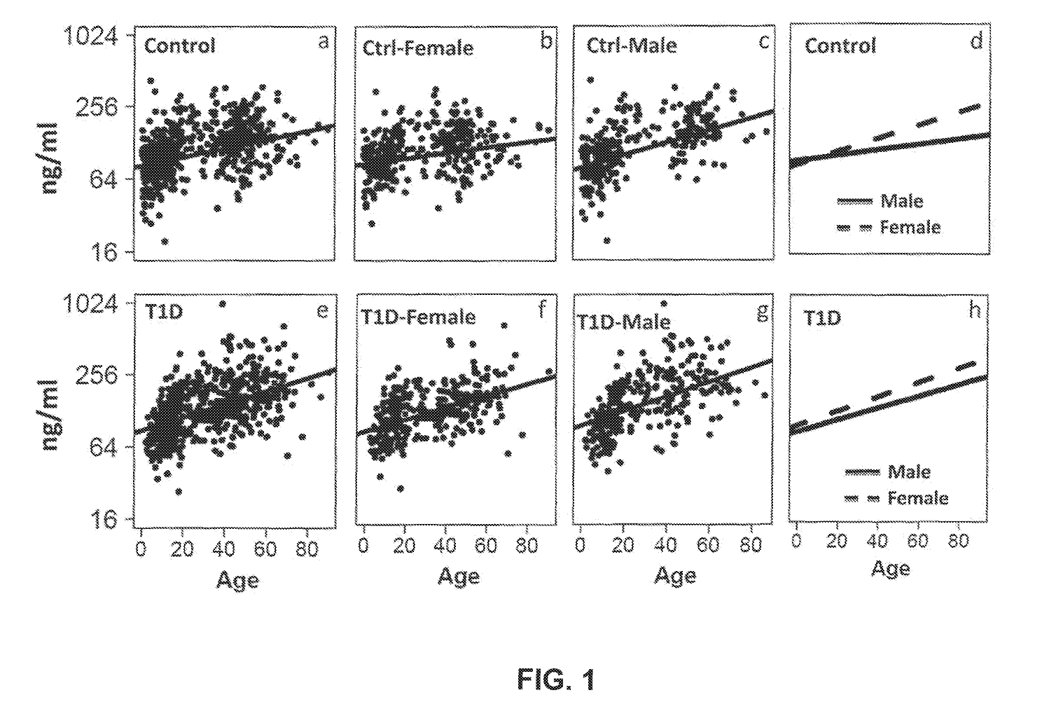

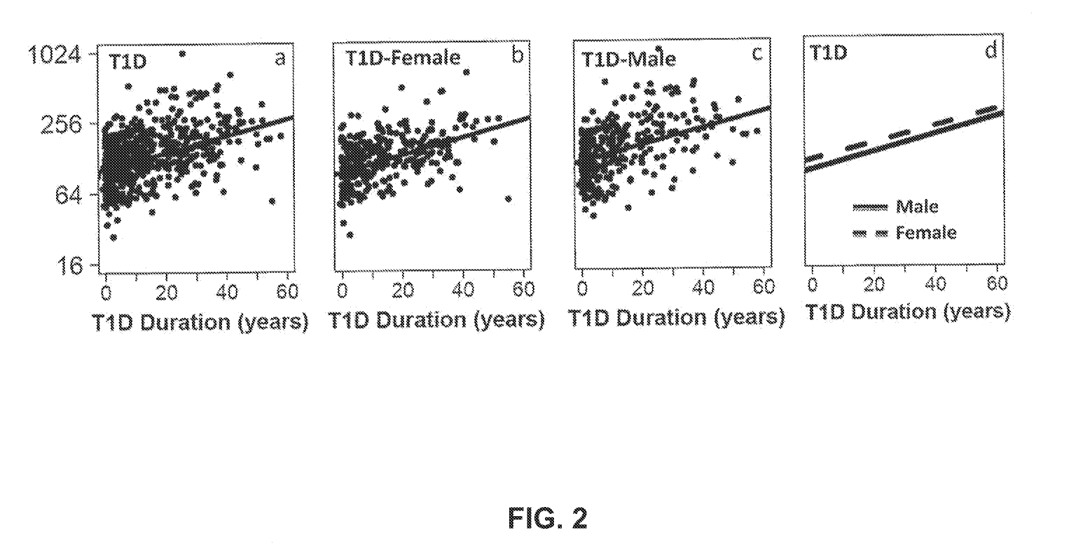

Regression analyses using IGFBP6 as dependent variable and age as a covariate indicated that IGFBP6 levels were significantly associated with age in both controls (r=0.22, p<10⁻⁹, FIG. 1a) and patients (r=0.37, p<10⁻¹⁶, FIG. 1e). Further regression analyses after stratification on gender and disease status revealed that age effect on IGFBP6 levels differ slightly according to the sex of the subjects in both the controls (r=0.143 for females and r=0.359 for males, FIG. 1 a-d) and T1D patients (r=0.37 for female and r=0.399 for males, FIG. 1e-h). A similar analysis was performed to determine correlation between IGFBP6 levels and duration of diabetes in T1D patients. It was found that IGFBP6 levels were significantly correlated with duration of diabetes in both females (r=0.35, p<10⁻¹², FIG. 2b) and males (r=0.35, p<10⁻¹¹, FIG. 2c). Sex-specific differences in the correlation of IGFBP6 levels with duration of diabetes (FIG. 2d) was not observed. Since IGFBP6 levels were significantly associated with age and sex of the study subjects, controls and patients for these two variables were matched to create case-control matched dataset for further analyses.

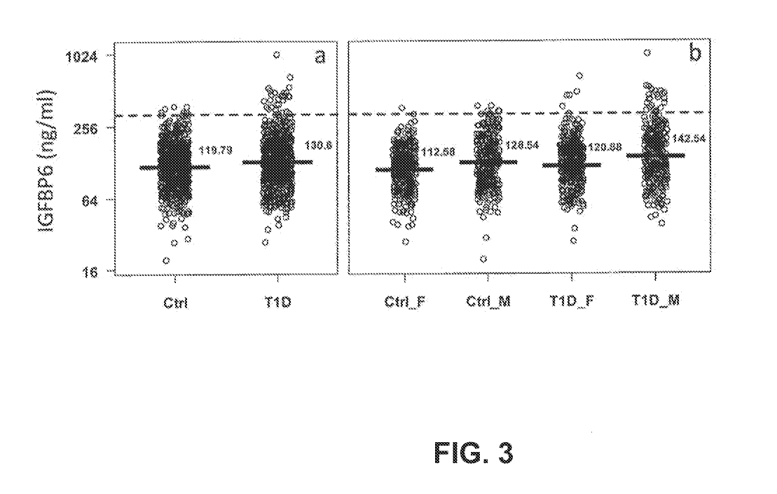

The mean IGFBP6 levels were slightly but significantly higher in the T1D group (mean=130.7 ng/ml) compared with the control group (mean=119.7 ng/ml) (p<10⁻⁵, FIG. 3a). When data was stratified by sex, the differences between T1D and control subjects were significant in both male (T1D mean 142.5 vs control mean 128.54 ng/ml, p<0.05) and female (T1D mean 120.8 vs control mean 112.6 ng/ml, p<0.05) (FIG. 3b). Sex-specific differences in serum levels of IGBP6 in both controls (males=128.5 ng/ml and females=112.6 ng/ml, FIG. 3b) and T1D patients (males=142.5 ng/ml and females=120.9 ng/ml, FIG. 3b) were also observed. Interestingly, 4.1% (27/657) of patients with T1D had very high IGFBP6 levels (defined as above the 99^{th} percentile value in controls, 321.2 ng/ml) compared to 1% (7/670) of the controls (odds ratio=3.99, p<10⁻⁴) (FIG. 3b).

The association between IGFBP6 concentrations and diabetes risk was also examined using conditional logistic regression. Odds ratio (OR) of diabetes per SD increment of protein levels were computed. First, the serum levels of IGFBP6 were treated as continuous variable and regression was performed with disease as a categorical variable. A significant association of IGFBP6 concentration with increased risk of T1D (OR=1.26, p<0.01, Table 2) was observed. To assess the relative risk of diabetes across different quartiles of IGFBP6 concentrations, the serum levels of IGBP6 from controls were used at the 25^{th}, 50^{th} and 75^{th} percentile as cutoff values to categorize T1D subjects into four quartiles. There are significantly more T1D subjects in the top quartile than in the 1st quartile (OR=1.69, Table 2).

TABLE 2
Relative risk (odds ratios) of T1D and its complications with increase in IGFBP6 levels

| Groups⁺ | N | OR | pval | OR2_Q2 | OR3_Q3 | OR4_Q4 | p-trend |
|---|---|---|---|---|---|---|---|
| T1D | 696 | 1.26 | 0.0008 | 1.35 | 0.94 | 1.69 | 0.06 |
| | | (1.10-1.44) | | (0.97-1.89) | (0.64-1.37) | (1.18-2.43) | |
| Nephropathy | 36 | 4.03 | 0.002 | 0.67 | 2.13 | 22.91 | 0.06 |
| | | (1.67-9.70) | | (0.13-3.42) | (0.52-8.68) | (2.44-215.1) | |
| Photocoagulation | 55 | 1.94 | 0.021 | 0.63 | 1.54 | 3.64 | 0.32 |
| | | (1.10-3.41) | | (0.19-2.01) | (0.49-4.85) | (1.13-11.68) | |
| Retinopathy | 73 | 1.66 | 0.042 | 0.84 | 1.76 | 2.32 | 0.35 |
| | | (1.02-2.70) | | (0.33-2.11) | (0.70-4.42) | (0.88-6.07) | |
| Blindness | 15 | 6.93 | 0.081 | ND | ND | ND | ND |
| | | (0.79-60.74) | | | | | |
| Foot Ulcer | 10 | 4.32 | 0.144 | ND | ND | ND | ND |
| | | (0.61-30.78) | | | | | |
| CAD | 35 | 1.51 | 0.253 | 1.65 | 0.88 | 2.07 | 0.43 |
| | | (0.75-3.05) | | (0.52-5.2) | (0.25-3.18) | (0.51-8.35) | |
| HTN | 96 | 1.43 | 0.061 | 1.72 | 2.54 | 1.70 | 0.03 |
| | | (0.98-2.08) | | (0.86-3.44) | (1.23-5.25) | (0.81-3.58) | |
| Amputation | 8 | 1.98 | 0.392 | ND | ND | ND | ND |
| | | (0.41-9.48) | | | | | |
| Peripheral | 65 | 1.21 | 0.469 | 0.54 | 1.51 | 1.58 | 0.84 |
| Neuropathy | | (0.72-2.06) | | (0.21-1.38) | (0.59-3.92) | (0.58-4.32) | |
| N | 112 | 0.96 | 0.812 | 0.8 | 0.57 | 0.72 | 0.16 |
| | | (0.71-1.30) | | (0.44-1.44) | (0.31-1.06) | (0.37-1.38) | |

⁺T1D was compared to controls. Each category of complications was compared to T1D without any complications.
OR: Odds ratio. Values presented are OR and its 95% confidence interval.
Q2, Q3 and Q4: 2^{nd}, 3^{rd} and 4^{th} quartiles were compared to 1^{st} quartile (reference)
HTN: Hypertension, CAD: Coronary Artery Disease.
ND: not done due to low sample size.

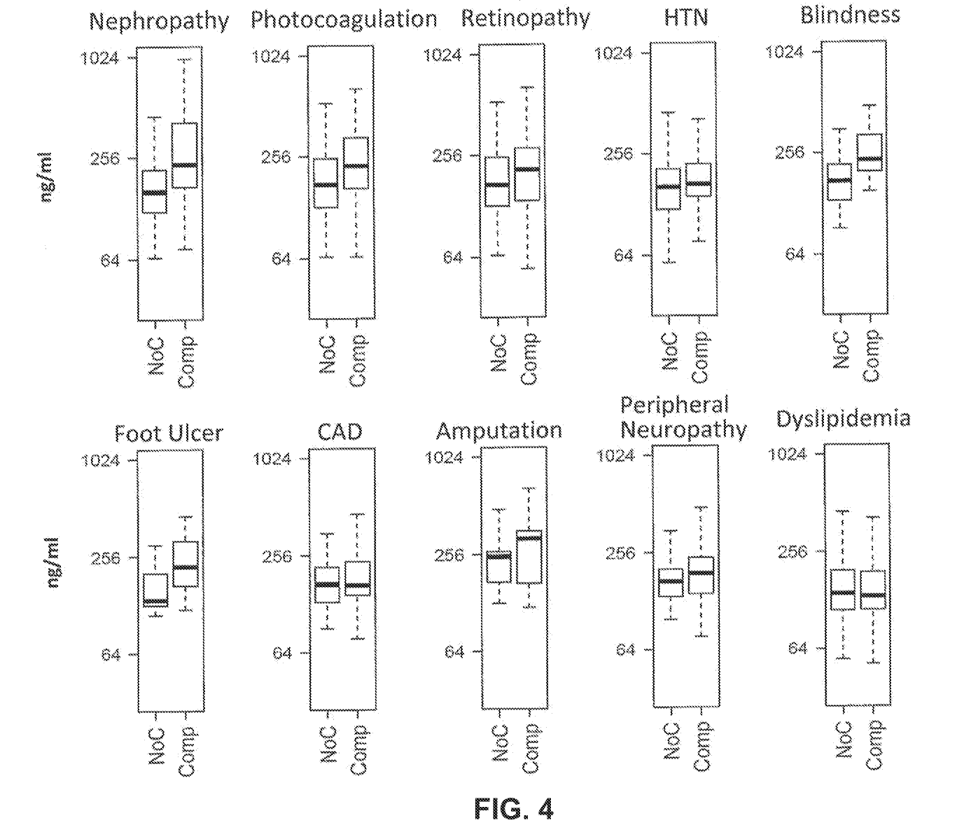

The association of IGFBP6 levels with different diabetic complications was also examined in T1D patients. For each complication, a matched pair dataset was created to alleviate the effect of age, sex and duration of disease. Each T1D patient with complication was matched to a T1D patient without any complication to create age, sex, and duration of diabetes matched pairs. The boxplots depicting distribution of IGFBP6 levels in patients with different complications are shown in FIG. 4. The mean levels of serum IGFBP6 were significantly increased (p<0.05) in T1D patients with nephropathy (n=36), photocoagulation (n=55), retinopathy (n=73), and blindness (n=15) compared to T1D patients without any complication (Table 3). Conditional regression analysis further revealed a significantly increased risk of nephropathy (OR=4.03, p<0.01), photocoagulation (OR=1.94, p<0.05) and retinopathy (OR=1.66, p<0.05) with per SD increase in IGFBP6 concentrations (Table 2). T1D patients with serum levels of IGFBP6 in 4^{th} quartile had a very high risk of developing nephropathy (OR=22.91), followed by T1D subjects who underwent photocoagulation to stall the diabetic retinopathy (OR=3.64) and retinopathy (OR=2.32) as compared to the 1^{st} quartile (Table 2).

TABLE 3
Serum IGFBP6 levels in T1D patients with and without diabetic
complications.

| Complication | N | No Comp | Comp | FC* | p-value |
|---|---|---|---|---|---|
| Nephropathy | 36 | 161.1 | 244.4 | 1.52 | 0.00014 |
| Photocoagulation | 55 | 177.9 | 225.6 | 1.27 | 0.01203 |
| Blindness | 15 | 174.9 | 257.3 | 1.47 | 0.02251 |
| Retinopathy | 73 | 174.7 | 203.2 | 1.16 | 0.03315 |
| HTN | 96 | 158.8 | 176.7 | 1.11 | 0.07622 |
| Foot Ulcer | 10 | 160.2 | 227.3 | 1.42 | 0.09269 |
| CAD | 35 | 151.5 | 183.2 | 1.21 | 0.16613 |
| Amputation | 8 | 220.6 | 261.8 | 1.19 | 0.52837 |
| Peripheral | 65 | 173.7 | 180.1 | 1.04 | 0.69107 |
| Neuropathy | | | | | |
| Dyslipidemia | 112 | 147.1 | 144.9 | 0.99 | 0.77589 |

*FC: Fold Change was calculated for each specific category of T1D with complication (Comp) over the T1D patients without any complication (No Comp).
HTN: Hypertension,
CAD: Coronary Artery Disease.

### Discussion

These results indicate that males in both controls and T1D group had higher levels of IGFBP6 as reported earlier [Yu H, et al. J Clin Lab Anal 1999; 13(4): 166-172]. Furthermore, T1D is associated with an increase in serum levels of IGFBP6 similar to the observations previously reported for IGFBP1, 2, 3 and 5. [Mohamed-Ali V, et al. Clin Endocrinol (Oxf) 1999; 50(2): 221-228; Sireesha M, et al. J Diabetes 2009; 1(2): 118-124; Rajaram S, et al. Endocr Rev 1997; 18(6): 801-831; Wheatcroft S B and Kearney M T. Trends Endocrinol Metab 2009; 20(4): 153-162]. Several IGFBPs have been measured in T1D patients with or without diabetic complications as well as controls in several previous studies [Mohamed-Ali V, et al. Clin Endocrinol (Oxf) 1999; 50(2): 221-228; Sireesha M, et al. J Diabetes 2009; 1(2): 118-124; Wheatcroft S B and Kearney M T. Trends Endocrinol Metab 2009; 20(4): 153-162]. These studies have established the role of these IGFBPs as regulators of obesity, insulin resistance, bone mineral density and complications such as retinopathy and nephropathy in T1D and T2D [Jehle P M, et al. Eur J Intern Med 2003; 14(1): 32-38]. These results demonstrate for the first time the relationship between T1D and serum levels of IGFBP6, previously demonstrated for other IGFBPs in T1D and pathophysiology of insulin-mediated metabolic dysfunction [Mohamed-Ali V, et al. Clin Endocrinol (Oxf) 1999; 50(2): 221-228; Ruan W and Lai M. Acta Diabetol 2010; 47(1): 5-14; Wheatcroft S B and Kearney M T. Trends Endocrinol Metab 2009; 20(4): 153-162]. Serum IGFP6 levels were measured in 697 T1D patients in different age groups and with/without diabetic complications. This large dataset allowed examination of the various parameters that may influence serum IGFBP6 levels in T1D patients. The results indicated that serum IGFBP6 levels differ by sex and increase with age as reported previously for other IGFBPs [van D J, et al. Clin Endocrinol (Oxf) 1999; 50(5): 601-609]. A positive association was also observed between duration of T1D and IGFBP6 levels in T1D patients, the slopes of the regression lines indicated that serum levels of IGFBP6 were associated with the disease duration for both genders.

Serum levels of IGFBP6 are elevated in diabetic patients. Elevation in serum levels is more prominent in males than females as observed in another disease [van D J, et al. Clin Endocrinol (Oxf) 1999; 50(5): 601-609]. These results are in complete agreement regarding serum levels of IGFBP 1 levels reported in T1D [Jehle P M, et al. Eur J Intern Med 2003; 14(1): 32-38]. The elevation in serum levels of IGFBP6 observed in these results can be explained by two ways. Firstly, glucose is reported to be the primary modulator of IGF-II production in pancreatic β-cells [Le Roith D. T. Exp Diabesity Res 2003; 4(4): 205-212] and uncontrolled hyperglycemia is known to cause elevation in IGF-II levels in T1D/T2D [Sireesha M, et al. J Diabetes 2009; 1(2): 118-124] and in cellular models. Furthermore, IGFs also stimulate hepatic and extra-hepatic production of IGFBPs in liver and regionally in tissues, indicating that the increase observed in this study may be mediated by IGF-II arising due to hyperglycemia. It has been known that metabolic conditions with decreased insulin levels such as fasting, diabetes and exercise causes increase in systemic levels of IGFBP and insulin is one of the suppressors of IGFBP production in liver [Rosenfel R. G. and Roberts C. T. The IGF system: molecular biology, physiology, and clinical applications. 1999]. In fact, patients with T1D have reduced levels of IGF in their serum and elevated levels of IGFBP1, especially in subjects with poor glycemic control or micro-vascular complications [Feldmann B, et al. Growth Horm IGF Res 2000; 10(1): 53-59].

Evidence is now emerging that dysregulation of the IGF system is involved in the development of complications of diabetes such as retinopathy, neuropathy, cardiovascular diseases and nephropathy [Jehle P M, et al. Eur J Intern Med 2003; 14(1): 32-38; Mohamed-Ali V, et al. Clin Endocrinol (Oxf) 1999; 50(2): 221-228; Sireesha M, et al. J Diabetes 2009; 1(2): 118-124; Feldmann B, et al. Growth Horm IGF Res 2000; 10(1): 53-59; Bayes-Genis A, et al. Circ Res 2000; 86(2): 125-130; Sima A A, et al. Exp Diabesity Res 2003; 4(4): 235-256]. IGFs, especially IGF-II have strong anti-apoptotic properties and are involved in proliferation of endothelial cells and several cancer cell lines. It has been argued that regional concentrations of IGFs may be more significant than systemic levels in the pathogenesis of diabetic complications [Livingstone C and Ferns G. A. Br J Diabetes Vasc Dis 2003; 3: 326-331]. IGFs, especially IGF-II, are known to exert its anti-apoptotic responses by binding to insulin receptor (IR) in many tissues. The cytoprotective effects of IGF-II are related to mitochondrial protection leading to increased ATP production reducing free radical generation, oxidative damage and apoptosis [Garcia-Fernandez M, et al. J Transl Med 2011; 9: 123; Castilla-Cortazar I, et al. J Transl Med 2011; 9: 103]. On the other hand, increased expression of IGF-II has been reported to be one of the contributors of diabetic nephropathy via excessive deposition of extra-cellular matrix components in the glomerular basement membrane [Sireesha M, et al. J Diabetes 2009; 1(2): 118-124]. The mean serum IGFBP6 levels are elevated in T1D patients with diabetic complications including nephropathy, blindness, retinopathy, photocoagulation and coronary artery disease (CAD), indicating an elevated suppression of IGF actions in these tissues/vascular compartments. A major function of IGFBP6 is to inhibit anti-apoptotic actions of IGF-II [Rajaram S, et al. Endocr Rev 1997; 18(6): 801-831]. The reduction in IGF-II levels via excessive binding to IGFBP6 indicates an enhancement of apoptosis in the selected organs.

Cell proliferation and differentiation is an important step in wound healing. IGF-II, due to its cell proliferative property, has been implicated in wound healing and/or tissue regeneration [Kratz G, et al. Scand J Plast Reconstr Surg Hand Surg 1994; 28(2): 107-112; Kratz G, et al. Exp Cell Res 1992; 202(2): 381-385]. These results on elevated IGFBP6 levels in diabetic foot ulcers indicates that excessive IGFBP6 reduces the bio-availability of IGF-II required for healing at the site of tissue damage. This loss of wound healing may lead to excessive foot ulcers and amputation in T1D individuals.

In summary, striking relationships between IGFBP6 concentrations and several important components of the insulin deficiency have been demonstrated in T1D patients. Although the cross-sectional nature of the study cannot distinguish the underlying cause from effect, the logistic regression data indicates that T1D patients with higher IGFBP6 are at an increased risk of developing complications of the kidney, eyes and vascular system. Since IGFBP6 is a modulator of IGF-II action, it is inferred that IGF-II is abnormally regulated in T1D both systemically and regionally at the tissue levels.

I claim:

1. A method of treating diabetes type 1 in a subject comprising
administering a composition to the subject in an amount effective to reduce serum levels of insulin-like growth factor binding protein 6 (IGFBP6) relative to serum levels of IGFBP6 of the subject prior to treatment, wherein the reduction in serum levels of IGFBP6 treats one or more symptoms of diabetes or diabetic complications.

2. The method of claim 1, wherein the diabetic complication is nephropathy.

3. A method of treating diabetes in a subject comprising
a) administering a composition to the subject in an amount effective to reduce serum levels of IGFBP6,
b) assaying serum levels of IGFBP6 of the subject after administration of the composition, and
c) discontinuing treatment if IGFBP6 serum levels are normal.

4. The method of claim 3, wherein normal IGFBP6 serum levels are those levels present in the subject prior to developing diabetes.

5. The method of claim 3, wherein normal IGFBP6 serum levels are those levels present in non-diabetic subjects.

6. A method of determining the severity or monitoring of diabetes in a subject, the method comprising:
assaying for levels of IGFBP6 in a sample from the subject, wherein an increase in IGFBP6 levels in the sample compared to a control level is an indication that the subject will have or is having diabetic complications.

7. The method of claim 6, further comprising administering a therapeutic to the subject in an effective amount to treat the diabetes or a diabetic complication.

8. The method of claim 6, wherein the control levels is the IGFBP6 level present in a non-diabetic subject.

9. A method of determining the risk of diabetes in a subject, the method comprising:
assaying for levels of insulin-like growth factor binding protein 6 (IGFBP6) in a sample from the subject, wherein an increase in IGFBP6 levels in the sample compared to a control level is an indication that the subject is at risk of developing type 1 diabetes.

10. The method of claim 9, further comprising administering to the subject at risk of developing type 1 diabetes a therapeutic to prevent the diabetes or delay the onset of diabetes.

11. The method of claim 10, wherein the therapeutic is an immunomodulator

12. A method of selecting a therapy for a subject diagnosed with diabetes, the method comprising assaying for the levels of IGFBP6 in a sample from the subject, wherein an increase in IGFBP6 levels in the sample compared to a control level is an indication that a therapy comprising treating nephropathy is selected.

13. A method of identifying an agent for use in treating diabetes, comprising contacting a sample comprising IGFBP6 with a candidate agent and assaying for IGFBP6 in the sample, wherein a decrease in IGFBP6 in the sample compared to a control is an indication that the candidate agent is an effective agent for use in treating diabetes.

14. The method of claim 13, wherein a decrease in IGFBP6 is a decrease in protein expression.
